(19) United States
(12) Patent Application Publication
(10) Pub. No.: US 2008/0108826 A1
(43) Pub. Date: May 8, 2008

(54) PREPARATION OF 4-ALKOXY-7-(TETRAHYDRO-PYRAN-4-YL)-BENZOTHIAZOL-2-YLAMINE DERIVATIVES

(76) Inventor: Paul Spurr, Riehen (CH)

Correspondence Address: HOFFMANN-LA ROCHE INC.;PATENT LAW DEPARTMENT, 340 KINGSLAND STREET, NUTLEY, NJ, 07110, US

(21) Appl. No.: 11/926,787

(22) Filed: Oct. 29, 2007

(30) Foreign Application Priority Data
Nov. 3, 2006 (EP) 06123414.2

(51) Int. Cl. C07D 277/62; C07D 315/00

(52) U.S. Cl. 548/161; 549/427

(57) ABSTRACT

Processes for the preparation of 4-alkoxytetrahydropyrane derivatives used in the preparation of 4-alkoxy-7-(tetrahydropyran-4-yl)-benzothiazol-2-ylamine and derivatives.

### PRIORITY TO RELATED APPLICATION(S)

This application claims the benefit of European Patent Application No. 06123414.2, filed Nov. 3, 2006, which is hereby incorporated by reference in its entirety.

### BACKGROUND OF THE INVENTION

Compounds of formula I are important intermediates for the preparation of a number of pharmaceutically active ingredients, for example in the preparation of adenosine receptor ligands of formula

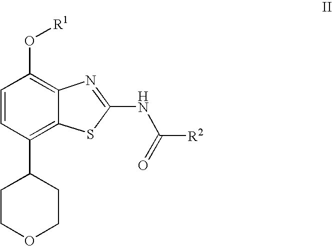

wherein R² is for example 7-oxabicyclo[2.2.1]hept-2-ylamine. Such compounds are disclosed in WO05000842.

### SUMMARY OF THE INVENTION

The present invention provides a process for the manufacture of compounds of the general formula

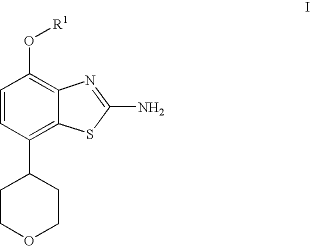

wherein

### R¹ is alkyl.

The process according to the invention is a short, cost effective route to compounds of formula I, involving the two key intermediates

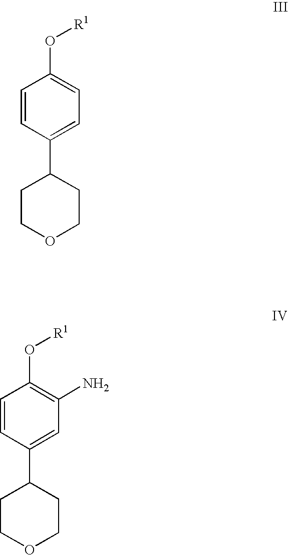

wherein R¹ is as defined above.

### DETAILED DESCRIPTION OF THE INVENTION

The process according to the invention is a short, cost effective route to compounds of formula I, involving the two key intermediates

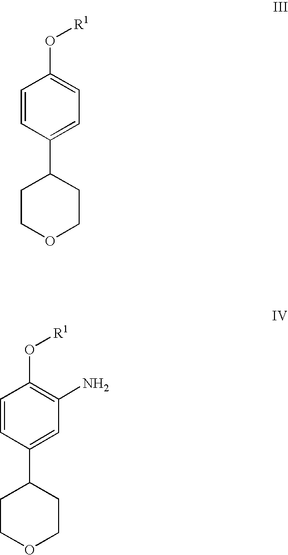

wherein R¹ is as defined above.

Compounds of formula III are new and therefore part of the invention. These compounds are prepared as depicted in Scheme 1 by the coupling of p-bromoalkoxy-benzene (1) and tetrahydropyran-4-one (2) in a Grignard reaction. Depending upon the reaction conditions, either 4-(4-alkoxy-phenyl)-tetrahydropyran-4-ol (3) or the corresponding dehydrated product 4-(4-alkoxyphenyl)-3,6-dihydro-2H-pyran (4) or a mixture thereof is obtained. When the reaction is conducted in the absence of acid, an excess of the 4-(4-alkoxyphenyl)-tetrahydropyran-4-ol (3) is obtained, the olefin (4) can be obtained by acid catalyzed dehydration of (3).

The compound 3 is reduced to 4-(4-alkoxyphenyl)-tetrahydropyran (III) with H₂ in the presence of a catalyst such as Pd/C and an acid, such as hydrochloric acid, in a solvent, such as ethanol. 4-(4-Methoxy-phenyl)-tetrahydropyran (III) can also be obtained from 4-(4-alkoxyphenyl)-3,6-dihydro-2H-pyran (4) by hydrogenation in the presence of Pd/C in an inert solvent, such as toluene.

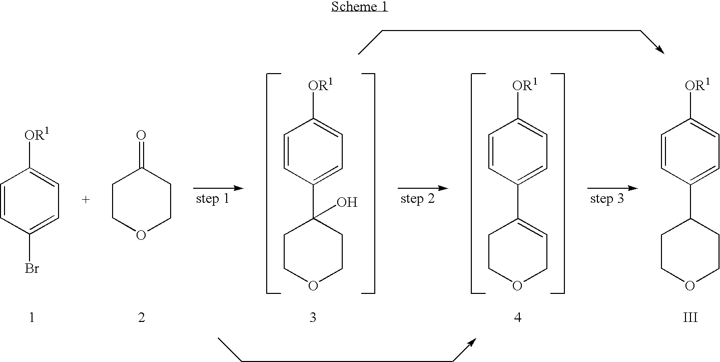

wherein

### R¹ is as defined above.

In the alternative approach, compounds of formula III are prepared starting from 4-alkoxybenzaldehyde (5) and an acetoacetyl derivative as depicted in Scheme 2

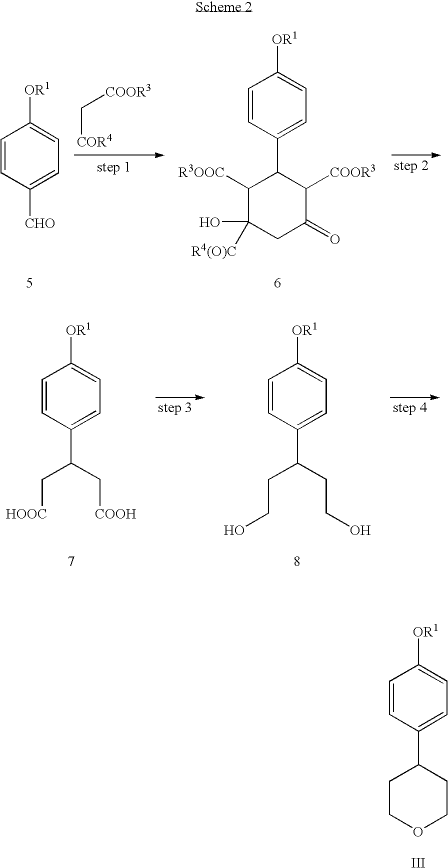

wherein

### R¹ is as defined above, and

### R³ and R⁴ are independently of each other alkyl.

Steps 1 and 2 are known in the art and can be carried out in analogy to the methods described in J. Org. Chem. 1971, 36(10), 1360-1365. The alkoxy aldehyde is converted to 3-the glutaric acid derivative (6) via a Knoevenagel reaction with ethyl acetoacetate followed by basic cleavage (a reverse Claisen reaction) which yields the acid (7).

According to the invention, the acid (7) is reduced, without previous esterification, with lithium aluminium hydride in tetrahydrofurane or 1,2-dimethoxyethane to the diol (4). Other reductive agents such as BH₃ or Red-Al® (sodium dihydro-bis-(2-methoxyethoxy)-aluminate solution) in an inert solvent such as diethylene glycol dimethyl ether (diglyme) are possible alternatives to LAH (lithium aluminium hydride) (Scheme 3).

Methods for the cyclization of diols are known in the art and described for example in J. Chem. Research (S) (1999) 326-327, Bull. Chem. Soc. Jpn (1980) 53, 3031-3032 and Synth. Comm (1987) 17(11), 1373-1376 and J. of Molecular Catalysis A: Chemical (1996) 149-155. However, it has been found that the cyclization of the diol of formula (8) proceeds smoothly and in high yield when performed in the presence of a specific type of zeolite catalyst, namely a zeolite with a SiO₂/Al₂O₃ ratio of 25-60 and Na₂O [wt %]>1 (Scheme 3).

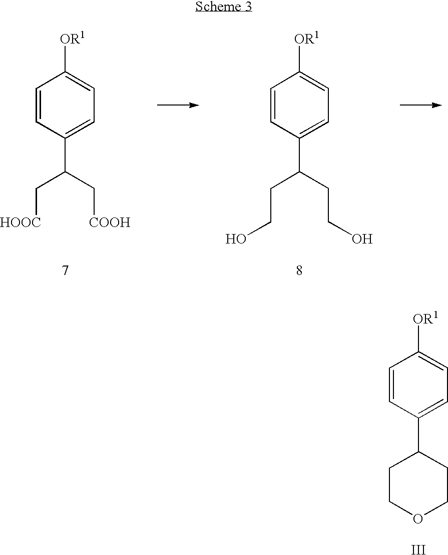

wherein

### R¹ is alkyl

A further embodiment of the present invention is thus the preparation of the a compound of formula III by cyclization of 3-(4-alkoxyphenyl)-pentane-1,5-diol (8) with the aid of a zeolite catalyst with a SiO₂/Al₂O₃ ratio of 25-60 and Na₂O [wt %]>1 in an inert solvent having a boiling point for the appropriate reaction temperature such as chlorobenzene.

Compounds of formula III are precursors of the second key intermediate, namely 2-methyl-5-(tetrahydropyran-4-yl)-phenylamine IV. A method for the preparation of such compounds starting with 4-bromo-2-nitroalkoxybenzene is given in WO 05/000842. This method, however, is not suitable for scale-up. It was therefore necessary to find alternative routes.

Compounds of formula IV are best prepared either by bromination of a compound of formula III and subsequent amination or optionally, by the direct nitration of the compound of formula III and reduction of the nitro-group.

The bromination of compound III is performed eg. with N-bromosuccimide in a solvent such as acetonitrile, acetic acid, ethylacetate, tetrahydrofurane or dichloromethane.

The conversion of 4-(4-alkoxy-3-bromophenyl)-tetrahydropyran (9) to the corresponding 2-alkoxy-5-(tetrahydropyran-4-yl)-phenylamine derivative (10) is carried out by catalytic amination in the known art with benzylamine or other suitable amine, such as diphenylmethylamine (Ph₂CHNH₂), tritylamine (Ph₃CNH₂), t-butylamine, allylamine or diallylamine, in the presence of a Pd-catalyst, such as tris(dibenzylidene acetone)dipalladium, and a ligand, such as rac-BINAP (2,2′-bis-(diphenylphosphino)-1,1′-binaphthalene), a base, such as sodium tert.-butylate, in a solvent, such as toluene. The amine protecting group is removed by catalytic methods well known in the art to form the desired amine of formula IV (Scheme 4).

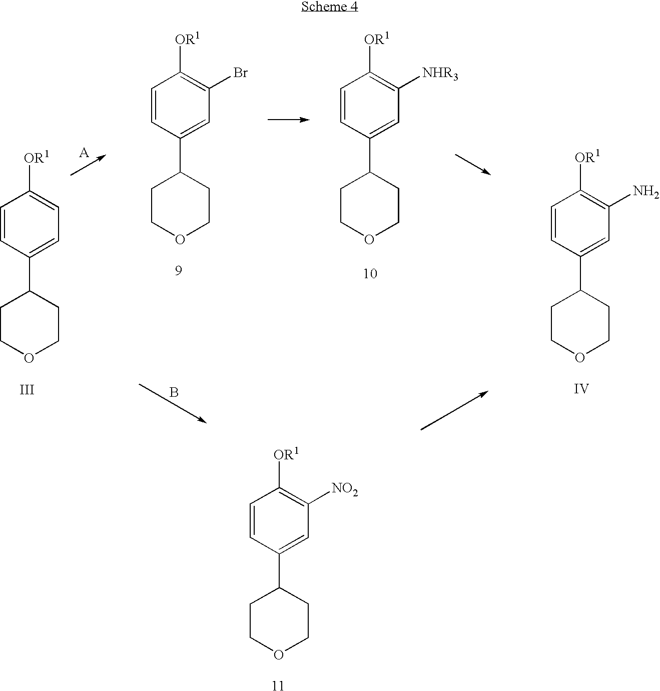

wherein

### R¹ is alkyl, and

### R³ is an amino protecting group, such as benzyl, trityl, t-butyl, allyl or diallyl.

A further embodiment of the present invention is thus the preparation of compounds of formula IV by a process which comprises

a) bromination of a compound of formula III with N-bromosuccimide in a solvent, such as acetonitrile, acetic acid, ethylacetate, THF or dichloromethane;
b) catalytic amination of the bromoderivative (2) with benzylamine or other suitable amine, such as diphenylmethylamine (Ph₂CHNH₂), tritylamine (Ph₃CNH₂), t-butylamine, allylamine or diallylamine, in the presence of tris(dibenzylidene acetone)dipalladium as a catalyst, rac-BINAP (2,2′-bis-(diphenylphosphino)-1,1′-binaphthalene) as a ligand, and sodium tert.-butylate as base in a solvent, such as toluene; and
c) deprotection of the amine protecting group by catalytic methods well known in the art, for example by hydrogenolysis in the presence of a metal catalyst, such as Pd/C.

In the alternative approach, compounds of formula IV are prepared by nitration of the compound of formula III. Nitration of alkoxy-derivatives, such as 4-(4-alkoxyphenyl)-tetrahydropyran, under standard conditions, such as HNO₃, yield primarily 4-(4-hydroxy-3-nitrophenyl)-tetrahydropyran which have to be realkylated. Nitration of 4-(4-alkoxyphenyl)-tetrahydropyran can be selectively achieved in the presence of acetyl nitrate, which is prepared in situ.

According to the invention, the nitration is carried out by reacting 100% HNO₃ in an appropriate solvent, such as dichloromethane, with acetic acid anhydride to form the nitration agent acetyl nitrate and adding the 4-(4-alkoxy-phenyl)-tetrahydropyran to the reagent; or by adding potassium nitrate and acetic acid anhydride to methanesulphonic acid in dichloromethane and subsequently adding 4-(4-alkoxyphenyl)-tetrahydropyran (III). Both reactions are carried out at a temperature of 0-20° C.

The reduction of the nitro derivative (11) can be performed according to methods well known in the art, for example by catalytic hydrogenation in the presence of metal catalyst, such as Pd/C.

Compounds of formula II can be prepared according to the method described in WO 01/97786 and US 2004/0138465 by thioacylation of compounds of formula IV with benzoylisothiocyanate, prepared in situ, to form the benzoylthiourea derivative (12), followed by hydrolysis under basic conditions and cyclization of compounds (13) with a sulphoxide/HBr/solvent combination as depicted in Scheme 5.

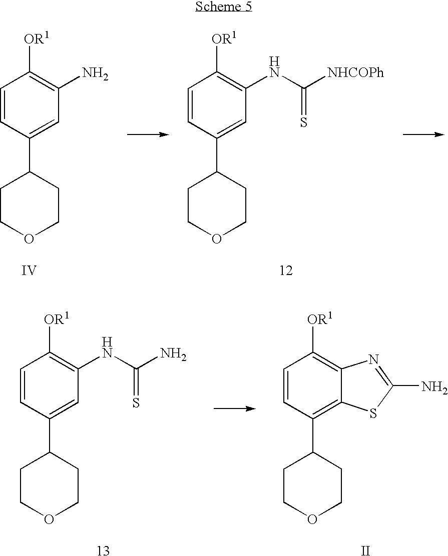

wherein

### R¹ is alkyl and Ph is phenyl.

While the above-mentioned method (US 2004/138465) is suited for compounds having a morpholino-group instead of a tetrahydropyranyl-group in position 5, it does not give sufficiently satisfactory results when used for the cyclization of N-[2-alkoxy-5-(tetrahydropyran-4-yl)-phenyl]-thioacetamide (13).

Cyclization proceeds in high yields when carried out in the presence of NH₄Br—H₂SO₄ (EP 529600). The reaction is performed by addition of a catalytic amount of aqueous ammonium bromide solution to the 2-alkoxy-5-(tetrahydropyran-4-yl)-phenylthioacetamide (13) in sulfuric acid.

In a further embodiment the invention is thus concerned with a process for the preparation of compounds of formula II which comprises the cyclization of a compound of formula (13) in the presence of NH₄Br—H₂SO₄.

According to the methods described in WO 01/097786, further reaction of compounds of formula II after activation, with for example 7-oxabicyclo[2.2.1]hept-2-ylamine yields the adenosine receptor ligands of formula of formula IIa (Scheme 6).

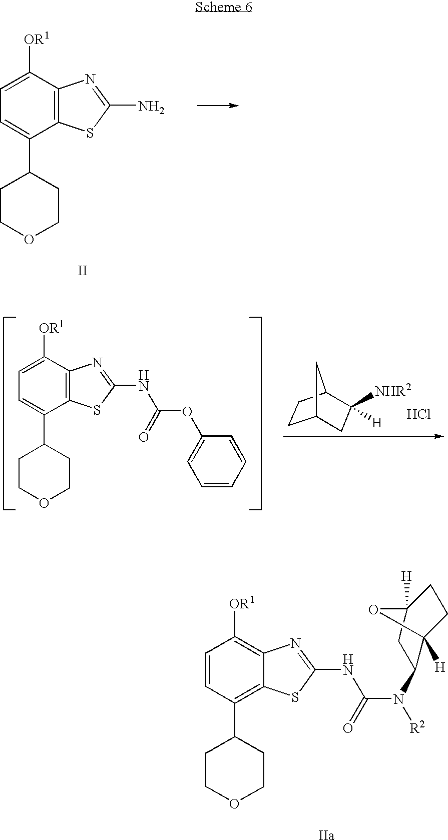

wherein

### R¹ is alkyl

### R² is alkyl

As used herein, the term “alkyl” denotes a saturated straight- or branched-chain alkyl group containing from 1 to 8 carbon atoms, for example, methyl, ethyl, propyl, isopropyl, n-butyl, i-butyl, 2-butyl, t-butyl and the like. Preferred lower alkyl groups are groups with 1-4 carbon atoms.

The term “amino protecting group” denotes any amine protecting group which is removable by hydrogenation or through isomerization. Such protecting groups are well known in the art, for example benzyl, diphenylmethyl (Ph₂CH—), trityl (Ph₃C—), tert.-butyl, allyl or diallyl.

Additional objects, advantages, and novel features of this invention will become apparent to those skilled in the art upon examination of the following examples thereof, which are not intended to be limiting.

### EXAMPLE 1

### a) Preparation of 4-(4-methoxyphenyl)-tetrahydropyran-4-ol (3)

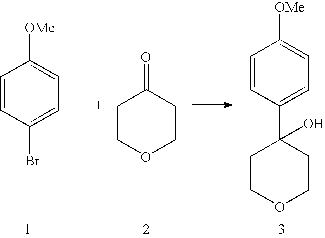

A 200 mL 4-necked round bottom flask equipped with reflux condenser, Ar adaptor, mechanical stirrer and thermometer was charged with 0.56 g magnesium turnings and 5 mL tetrahydrofurane. After the addition of 3 drops of 1,2-dibromoethane and some iodine crystals to activate the metal, 4.24 g bromoanisole (1) in 5 mL tetrahydrofurane were added over 10 min at a temperature of 50° C. Stirring was continued for 1 h to prepare the Grignard reagent. The reaction mixture was cooled to 0-5° C., diluted with 15 mL toluene and 2.19 mL 4-tetrahydropyran-2-one (2) in 5 mL toluene were added over 10 min. After stirring at 0-5° C. for 2 h, then at room temperature for 0.5 h, the reaction mixture was quenched by the addition of 1.33 mL acetic acid, 60 mL ethyl acetate and 60 mL water. The aqueous layer was separated and extracted with 60 mL ethylacetate. The organic layers were combined, washed with 60 mL saturated NaHCO₃ solution and with 60 mL water then dried over sodium sulphate. The suspension was filtered and the solvent evaporated yielding 4.58 g of 4-(4-methoxyphenyl)-tetrahydropyran-4-ol (3) as yellow green crystals.

¹H-NMR: (300 MHz, D₆-DMSO): δ=1.50 (bd, 2H, ax CH₂), 1.91 (dt, 2H, eq CH₂), 3.70 (m, 4H, CH₂O), 3.70 (s, 3H, OCH₃), 4.90 (s, 1H, OH), 6.87 (d, 2H, ArH), 7.38 (d, 2H, ArH)

MS: 208.2 (M⁺)

### EXAMPLE 1b

### Preparation of 4-(4-methoxyphenyl)-tetrahydropyran (III)

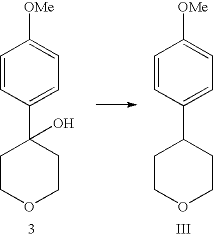

A 200 mL 4-necked round bottom flask equipped with reflux condenser, stirrer and thermometer was charged with 9.51 g 4-(4-methoxyphenyl)-tetrahydropyran-4-ol (3) in 100 mL ethanol. 1 mL 1N Hydrochloric acid and 475 mg 10% Pd/C were added and the reaction mixture was stirred at room temperature under a hydrogen atmosphere. After 4 h, a further 0.5 mL 1N hydrochloric acid and 200 mg 10% Pd/C was added and the reaction mixture stirred for another 1 h. The reaction was completed at 50° C. for 30 min. The reaction mixture was cooled and filtered over Dicalite. The solution was concentrated yielding 7.94 g 4-(4-methoxyphenyl)-tetrahydropyran (III) as a dark red oil.

¹H-NMR: (300 MHz, CDCl₃): δ=1.78 (m, 4H, CH₂), 2.69 (m, 1H, CH), 3.50 (m, 2H, ax CH₂O), 3.87 (s, 3H, OCH₃), 4.08 (m, 2H, eq CH₂O), 6.85 (d, 2H, ArH), 7.14 (d, 2H, ArH)

MS: 192.2 (M⁺)

### EXAMPLE 2

### Preparation of 4-(4-methoxyphenyl)-dihydropyran (4)

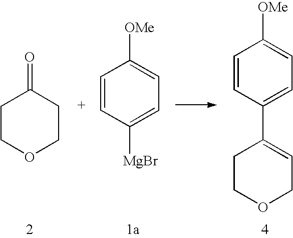

A 1500 mL 4-necked round bottom flask equipped with reflux condenser, Ar adaptor, stirrer and thermometer was charged with 1000 mL 0.5M 4-methoxyphenylmagnesium bromide (1a) (Aldrich, 0.5 M, 100%) solution and cooled to 0-2° C. 49.00 g Tetrahydro-4-pyranone (2) in 50 mL tetrahydrofurane were added at a temperature of 0-5° C. The reaction mixture was stirred for 45 min at 0-5° C. then the reaction was quenched by the addition of 17.95 mL 95% sulfuric acid diluted in 180 mL water. The temperature increased to 65° C., the reaction mixture was stirred for a further 45 min then cooled to 40° C. 100 mL Water was added and the solvent was exchanged under reduced pressure to 1000 mL toluene. The solution was washed twice with 600 mL water, 200 mL 5% sodium bicarbonate solution and 300 mL water. The aqueous layers were washed with 150 mL toluene, the organic layers combined, reduced to a volume of about 400 mL. Filtrated over 300 g silica gel 60 (0.063-0.200 mm) which was rinsed with toluene. The fractions containing the product were combined and reduced to a volume of about 400 mL estimated to contain ca. 93.11 g 4-(4-methoxyphenyl)-3,6-dihydro-2H-pyran (4). This solution was used directly for the next step. An small aliquot was evaporated, giving a yellow solid.

¹H-NMR: (300 MHz, CDCl₃): δ=2.50 (m, 2H, CH₂), 3.80 (s, 3H, OCH₃), 3.90 (t, 2H, CH₂O), 4.31 (dd, 2H, CH₂O), 6.03 (m, 1H, ═CH), 6.88 (d, 2H, ArH), 7.32 (d, 2H, ArH)

MS: 190.2 (M⁺)

### EXAMPLE 3

### Preparation of 4-(4-methoxyphenyl)-tetrahydropyran (III)

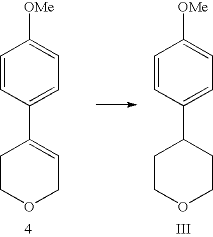

The above concentrate of ca. 93.11 g 4-(4-methoxyphenyl)-3,6-dihydro2H-pyran (4) in 300 mL toluene was charged in an autoclave. 11.40 g 10% Pd/C was added and the reaction mixture was hydrogenated with H₂ at 5 bar for 20 h at 20° C. The autoclave was flushed with argon, the reaction mixture filtrated and the filtrate evaporated yielding 72.0 g of 4-(4-methoxyphenyl)-tetrahydropyran (III) as a viscous oil.

### EXAMPLE 4

### Preparation of 4-(3-bromo-4-methoxyphenyl)-tetrahydropyran (9)

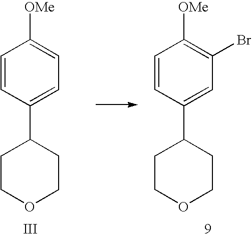

A 1500 mL 4-necked round bottom flask was charged with a suspension of 65.71 g N-bromosuccinimide in 500 mL acetonitrile. To this suspension, a solution of 71.20 g 4-(4-methoxyphenyl)-tetrahydropyran (III) (containing ca. 6% toluene) in 100 mL acetonitrile was added dropwise at room temperature. The reaction mixture was stirred for 4 h, evaporated and the residue was dissolved in 500 mL ethyl acetate and extracted twice with 300 mL water. The organic layer was dried over sodium sulphate and filtered. The filtrate was washed with ethyl acetate and the combined organic layers were evaporated providing a crystalline residue which was dissolved in 350 mL tert.-butylmethylether at 55° C. The solvent was exchanged continuously with 400 mL ethanol and the product crystallized after cooling the solution to 0-5° C. for 1 h. The suspension was filtered and the product was washed with 200 mL cold ethanol; yield 83.2 g 4-(3-bromo-4-methoxyphenyl)-tetrahydropyran (9) as white crystals.

¹H-NMR: (400 MHz, CDCl₃): δ=1.75 (m, 4H, CH₂), 2.70 (m, 1H, CH), 3.53 (m, 2H, ax CH₂O), 3.87 (s, 3H, OCH₃), 4.06 (m, 2H, eq CH₂O), 6.85 (d, 1H, ArH), 7.12 (dd, 1H, ArH), 7.40 (d, 1H, ArH)

MS: 270/272 (M⁺)

### EXAMPLE 5

### Preparation of benzyl-[2-methoxy-5-(tetrahydropyran-4-yl)-phenyl]-amine (10)

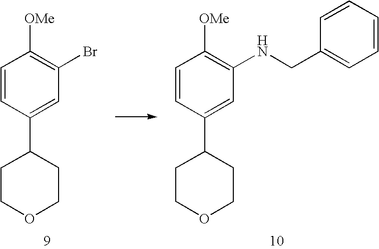

5.00 g 4-(3-Bromo-4-methoxyphenyl)-tetrahydropyran (9), 2.46 g sodium tert-butylate, 58.6 mg rac-BINAP and 33.8 mg tris(dibenzylidenacetone)dipalladium were charged in a reactor and dissolved in 40 mL toluene. The solution was stirred and heated to 100° C. and a solution of 2.13 g benzylamine in 9 mL toluene was added over 30 min. The reaction mixture was stirred for 30 min at 98° C. then cooled to 20° C. whereupon 1.55 g acetic acid in 29 g water were added, followed by another 30 mL water. The reaction mixture vigorously stirred for 1 min, then the layers were separated and the organic phase washed once more with 30 mL water. 25 mL Ethanol were added to the separated organic layer and the solution was concentrated under reduced pressure at 75° C. The oily residue was dissolved in 25 mL ethanol, concentrated to azeotropically remove remaining water and again dissolved in 25 mL ethanol. The resulting suspension was cooled to −15° C., the crystals were filtered and washed with 7.5 mL ethanol pre-cooled at −15° C. After drying at 50° C./11 bar for 1 h, 4.85 g benzyl-[2-methoxy-5-(tetrahydropyran-4-yl)-phenyl]-amine (10) was obtained as beige crystals.

¹H-NMR: (400 MHz, CDCl₃): δ=1.75 (m, 4H, CH₂), 2.62 (m, 1H, CH), 3.48 (dt, 2H, ax CH₂O), 3.82 (s, 3H, OCH₃), 4.03 (m, 2H, eq CH₂O), 4.35 (bs, CH₂N), 4.59 (bs, 1H, NH), 6.47 (d, 1H, ArH), 6.52 (dd, 1H, ArH), 6.72 (d, 1H, ArH), 7.30 (m, 5H, PhH)

MS: 298.3 (M+H⁺)

### EXAMPLE 6

### Preparation of 2-methoxy-5-(tetrahydropyran-4-yl)-phenylamine (IV)

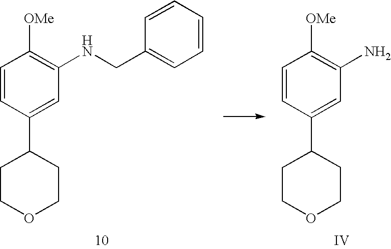

In a 500 ml autoclave flushed with argon, 26.5 g benzyl amine 10 was suspended in 106 mL ethanol at room temperature and 12.8 g methanesulfonic acid was slowly added followed by 2.22 g 10% Pd/C. The hydrogenolysis was performed under a pressure of 4 bar H₂ at 20° C. for 6 h. The reaction mixture was filtered, the residue was washed with ethanol and the filtrate was concentrated to about 50 mL under reduced pressure. After cooling to 25° C., 130 mL water was added to the residue followed by 67.25 g 2N sodium hydroxide. A precipitate formed and the pH of the slurry was further raised from 4.7 to 11.5. 300 mL Isopropyl acetate (300 mL) were added to reaction mixture and after stirring for 5 min, the layers were separated and the aqueous layer was washed with 170 mL isopropyl acetate. The combined organic layers were washed once with 20 mL water, the organic extracts were dried by azeotropic distillation and concentration of the solvent. 300 mL n-Heptane was added to the concentrate and remaining isopropyl acetate was removed by concentration. The temperature was increased to 95° C. to induce dissolution then lowered to 0° C. to complete crystallisation. The suspension was stirred for 1 h at 0° C., filtered, the product was washed twice with ice-cold n-heptane and dried at 50° C./30 mbar yielding 17.59 g 2-methoxy-5-(tetrahydropyran-4-yl)-phenylamine (IV).

¹H-NMR: (300 MHz, CDCl₃): δ=1.75 (m, 4H, CH₂), 2.62 (m, 1H, CH), 3.48 (m, 2H, ax CH₂O), 3.84 (s, 3H, OCH₃), 4.03 (m, 2H, eq CH₂O), 6.59 (dd, 1H, ArH), 6.63 (d, 1H, ArH), 6.74 (d, 1H, ArH)

MS: 298.3 (M+H⁺)

### EXAMPLE 7

### Preparation of 4-(4-methoxy-3-nitrophenyl)-tetrahydropyran (11)

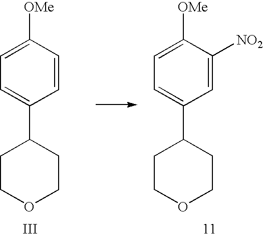

A 10 ml 3 necked round bottom flask was charged with 202.2 mg potassium nitrate in 1 mL dichloromethane and cooled to 0-5° C. 135.6 μL Methansulfonic acid were added and after 5 min, 274.1 μL acetic acid anhydride in 0.5 mL dichloromethane were added and stirred for 10 minutes. 192.3 mg 4-(4-Methoxyphenyl)-tetrahydropyran (III) in 1 ml dichloromethane were added. The reaction mixture was stirred for 2 h at 0-5° C., diluted with 10 ml dichloromethane, then washed with 10 ml water and 10 ml 1M NaHCO₃. The organic phase was dried over sodium sulphate, filtered and concentrated yielding 200 mg 4-(4-methoxy-3-nitrophenyl)-tetrahydropyran (11) as a yellow oil.

¹H-NMR: (400 MHz, CDCl₃): δ=1.77 (dt, 4H, CH₂), 2.77 (m, 1H, CH), 3.52 (m, 2H, ax CH₂O), 3.95 (s, 3H, OCH₃), 4.07 (dt, 2H, eq CH₂O), 7.05 (d, 1H, ArH), 7.40 (dd, 1H, ArH), 7.71 (d, 1H, ArH)

MS: 237.2 (M⁺)

### EXAMPLE 8

### Preparation of 2-methoxy-5-(tetrahydropyran-4-yl)-phenylamine (IV)

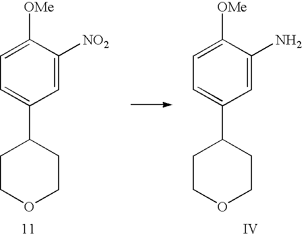

A 10 ml round bottom flask was charged with 192 mg 4-(4-methoxy-3-nitrophenyl)-tetrahydropyran (11) and 20.0 mg 10% Pd/C in 2 ml ethanol. The suspension was hydrogenated for 3 h at 50° C. under 1 bar H₂. The suspension was filtered and the solution concentrated yielding 160 mg 2-methoxy-5-(tetrahydropyran-4-yl)-phenylamine (IV) as brown crystals.

### EXAMPLE 9

### Preparation of 4-acetyl-4-hydroxy-2-(4-methoxyphenyl)-6-oxo-cyclohexane-1,3-dicarboxylic acid diethyl ester (6)

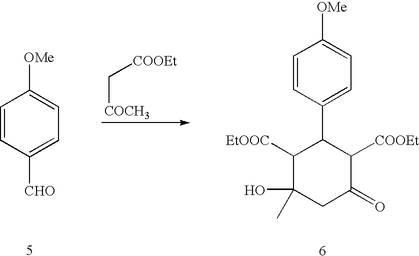

A 750 mL 4-necked round bottom flask equipped with reflux condenser, Ar adaptor, mechanical stirrer and thermometer was charged with 34 g 4-methoxybenzaldehyde (5) in 200 mL ethanol at ambient temperature. 65 g 3-Oxo-butyric acid ethyl ester was added, the funnel flushed with 20 mL ethanol and 4.2 mL piperidine was added over 10 min. After a total of 41 h, 220 mL n-hexane were added, the reaction suspension was cooled to 2° C. and stirred for another 1 h. The product (6) was filtered, washed three times with 25 mL n-hexane and dried at 45° C./25 mb for 2 h; yield 67.9 g 4-acetyl-4-hydroxy-2-(4-methoxyphenyl)-6-oxo-cyclohexane-1,3-dicarboxylic acid diethyl ester (6) as white crystals.

### EXAMPLE 10

### Preparation of 3-(4-methoxyphenyl)-pentanedioic acid (7)

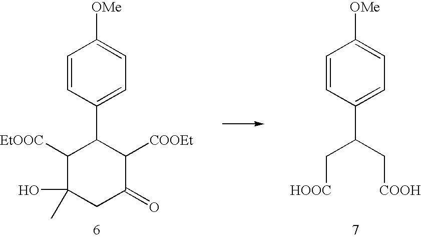

In a 750 mL 4-necked round bottom flask equipped with a mechanical stirrer, reflux condenser, thermometer and Ar adapter, 67.30 g 4-acetyl-4-hydroxy-2-(4-methoxyphenyl)-6-oxo-cyclohexane-1,3-dicarboxylic acid diethyl ester (6) was suspended in 150.0 mL ethanol. 150 mL 32% Sodium hydroxide solution was added over 10 min. The reaction mixture was heated to 80° C. and stirred for 2.5 h. After cooling to 20° C., the solvent was evaporated under reduced pressure, 350 mL water was added and the solution extracted twice with 150 mL tert.-butylmethyl ester. Residual ethanol in the aqueous layer was removed under reduced pressure on a rotavaporator. After acidification with 230 mL 25% hydrochloric acid, the suspension was stirred for 15 min at 2° C. to complete crystallization. The precipitate was filtered, washed three times with 150 mL water and dried at 50° C./15 mbar yielding 35.7 g 3-(4-methoxyphenyl)-pentanedioic acid (7) as a beige solid.

### EXAMPLE 11

### Preparation of 3-(4-methoxyphenyl)-pentane-1,5-diol (8) {replaced}

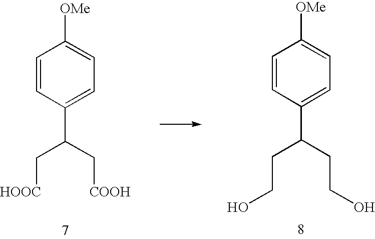

To 4.51 g lithium aluminiumhydride (LAH) in 50 mL THF, precooled to 0-5° C. was added a suspension of 16.64 g 3-(4-methoxyphenyl)-pentanedioic acid (7) in 175 mL THF. Stirring at 0-5° C. was continued for 1 h, at room temperature for 20 h then at reflux for 5 h. Additional 0.45 g LAH was added and after a further 2 h at reflux, the reaction was complete. The mixture was cooled to 0-5° C. and quenched with 5 mL water followed by 5 mL 4N sodium hydroxide and 15 mL water. The suspension was stirred at 0-5° C. for 1 h then filtered over dicalite which was rinsed twice with 50 mL THF. Evaporation of the filtrate provided 10.6 g 3-(4-methoxyphenyl)-pentane-1,5-diol (8) as a beige oil from which white crystals could be obtained with tert.-butylmethyl ether.

### EXAMPLE 12

### Preparation of 4-(4-methoxyphenyl)-tetrahydropyran (III)

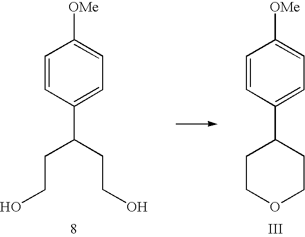

3.1 g ZEOcat® PB/H was added to a solution of 9.0 g 3-(4-methoxyphenyl)-pentane-1,5-diol (8) in 40 mL chlorobenzene. The reaction mixture was heated at 115° C. for 4 h then cooled to room temperature and filtered over 5 g dicalite. The filter pad was washed with tert.-butylmethyl ether, the filtrate concentrated and the residue dissolved in ethyl acetate. The solution was washed with 50 mL water, the layers separated and the organic phase was dried over 20 g sodium sulphate. Filtration then concentration afforded a residue which was dissolved in 150 mL ethanol. Evaporation azeotropically removed residual chlorobenzene and yielded 6.6 g of product (III) as a light yellow oil.

### EXAMPLE 13

### Preparation of 1-benzoyl-3-[2-methoxy-5-(tetrahydropyran-4-yl)-phenyl]-thiourea (12)

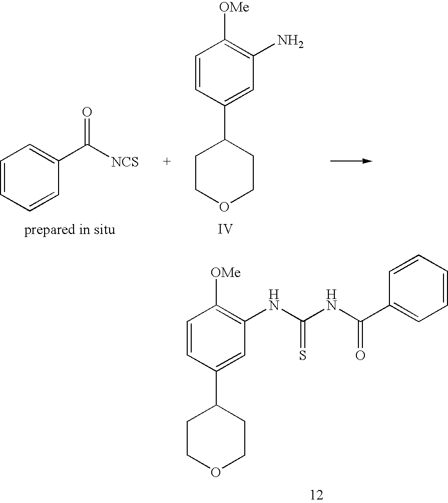

A 500 mL 4-necked round bottom flask equipped with reflux condenser, Ar adaptor, mechanical stirrer, addition funnel and thermometer was charged with 9.66 g ammonium thiocyanate dissolved in 145 mL acetone and heated to 45° C. 16.12 g Benzoylchloride was added over 30 min and the reaction mixture was heated to 55° C. and stirred for an additional 30 min. A solution of 21.6 g 2-methoxy-5-(tetrahydropyran-4-yl)-phenylamine (IV) in 145 mL warm (50° C.) acetone was added over 45 min and the reaction mixture was stirred and refluxed for 2 h. After evaporation of the solvent, the residue was distributed between 160 mL dichloromethane and 250 mL 5% sodium bicarbonate solution. The aqueous phase was separated and extracted with 50 mL dichloromethane. The combined organic phases were concentrated and the solvent continuously replaced by tert.-butylmethyl ether. The crystalline product was filtered and dried at 45° C./25 mb yielding 37.56 g 1-benzoyl-3-[2-methoxy-5-(tetrahydropyran-4-yl)-phenyl]-thiourea (12) as a yellow solid.

### EXAMPLE 14

### Preparation of 2-methoxy-5-(tetrahydropyran-4-yl)-phenyl]-thiourea (13)

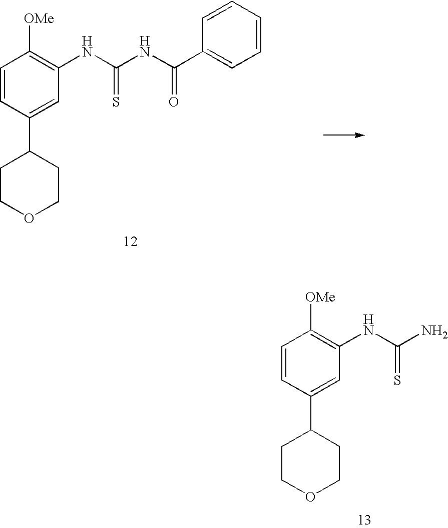

A 1000 mL reaction vessel equipped with reflux condenser, Ar adaptor, mechanical stirrer, addition funnel and thermometer was charged with 50.0 g 1-benzoyl-3-[2-methoxy-5-(tetrahydropyran-4-yl)-phenyl]-thiourea (12) dissolved in 420 mL methanol and 144 mL 4% sodium hydroxide solution was added over 15 min. The reaction mixture was stirred and heated at 65° C. for 2 h after which time 200 mL methanol was distilled and replaced by 300 mL water. The reaction mixture was concentrated to ca. 450 mL, cooled to ambient temperature and 400 mL dichloromethane were added. The aqueous phase was separated and extracted with 100 mL dichloromethane, the combined organic phases were washed with 200 mL water and the solvent replaced by 800 mL tert.-butylmethyl ether by distillation. The suspension formed was stirred at 0-5° C. for 2 h, filtered and the product was washed twice with 200 mL ice-cold tert.-butylmethyl ether. After drying at 45° C./25 mb, 33.81 g 2-methoxy-5-(tetrahydropyran-4-yl)-phenyl]-thiourea (13) was obtained as a light yellow solid.

### EXAMPLE 15

### Preparation of 2-amino-7-(tetrahydropyran-4-yl)-benzothiazol-4-ol (II)

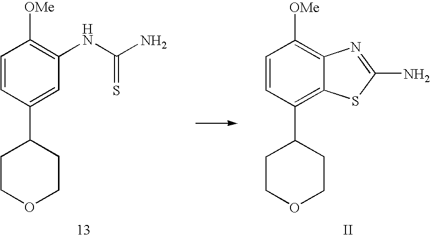

In a 4-necked round bottom flask equipped with reflux condenser, Ar adaptor, mechanical stirrer, addition funnel and thermometer, 63.0 g 2-methoxy-5-(tetrahydropyran-4-yl)-phenyl]-thiourea (13) were added to 125 mL sulfuric acid at 50° C. The reaction solution was heated to 65° C. and 2.32 g ammonium bromide in 6 mL water was added over 30 min so that the temperature remained below 70° C. After 5 min, the reaction mixture was cooled to room temperature and added to 650 ml 50% aqueous ethanol. The pH of the reaction mixture was adjusted to 9-10 by the addition of 360 ml 25% ammonium hydroxide. The suspension was stirred overnight at room temperature and then filtered. The product was washed with 60 ml 50% aqueous ethanol and 200 ml water then dried at 60° C./10 mbar providing 61 g 2-amino-7-(tetrahydro-pyran-4-yl)-benzothiazole (II) as a white solid.

¹H-NMR: (400 MHz, D₆-DMSO): δ=1.73 (m, 4H, CH₂), 2.67 (m, 1H, CH), 3.45 (m, 2H, ax CH₂O), 3.81 (s, 3H, OCH₃), 3.96 (m, 2H, eq CH₂O), 6.79 (d, 1H), 6.84 (d, 1H), 7.36 (bs, 2H)

MS: 265 (M+H⁺)

1. A process for the preparation of a compound of formula III 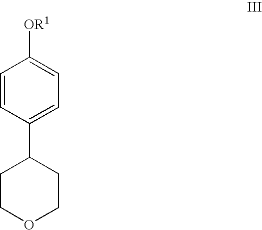
wherein R¹ is alkyl
which comprises
a) reduction of a diacid of formula 7 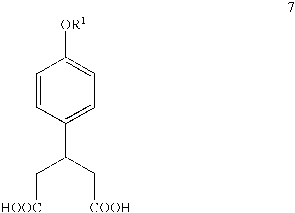
to form a compound of formula 8 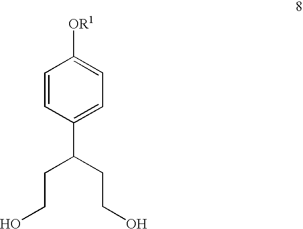
and b) subsequent cyclization the diol of formula (8)

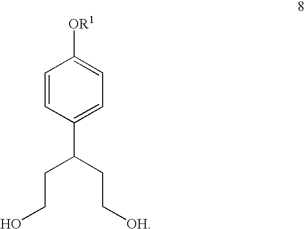

2. The process of claim 1, wherein the cyclization of a compound of formula 8 proceeds in the presence of a zeolite having an SiO₂/Al₂O₃ ratio of 25-60 and Na₂O [wt %] greater than 1.

3. A compound of formula III 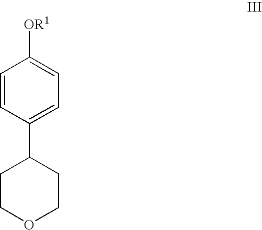
wherein R¹ is alkyl.

4. A process for the preparation of a compound of formula IV 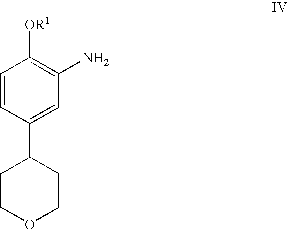
wherein R¹ is alkyl
which process comprises
a) reacting 100% HNO₃ in a solvent with acetic acid anhydride to form acetyl nitrate or by reacting potassium nitrate and acetic acid anhydride with methanesulphonic acid in dichloromethane and
b) adding a compound of formula III 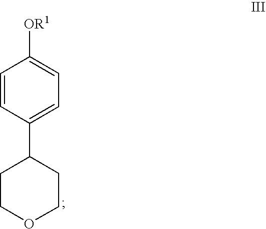
and subsequent
c) catalytic hydrogenation in the presence of a metal catalyst.

5. The process of claim 4 wherein the solvent is dichloromethane.

6. The process of claim 4 wherein the metal catalyst is Pd/C.

7. A process for the preparation of a compound of formula IV, 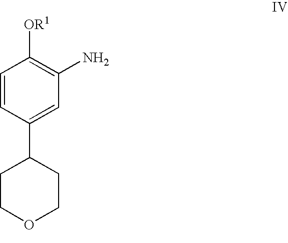
wherein R¹ is alkyl,
which comprises
a) bromination of a compound of formula III 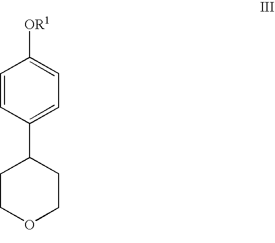
to produce a bromoderivative (9) 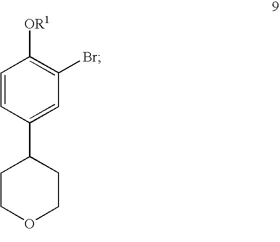
b) catalytic amination of the bromoderivative (9) 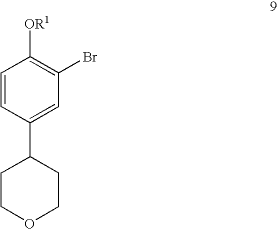
to form a compound of formula 10 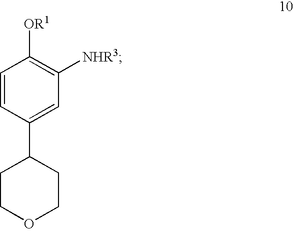
c) deprotection of the amine group in the compound of formula 10 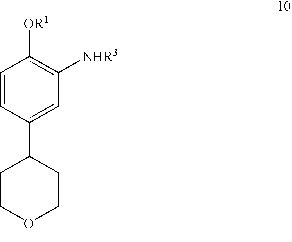
wherein R¹ is alkyl and R³ is benzyl, diphenylmethyl, trityl, t-butyl, allyl or diallyl.

8. A process for the preparation of a compound of formula II, 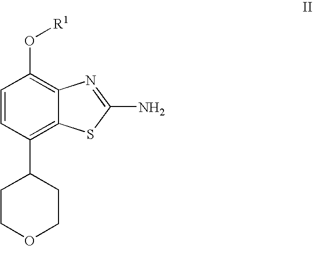
wherein R¹ is alkyl
which comprises cyclization of a compound of formula 13 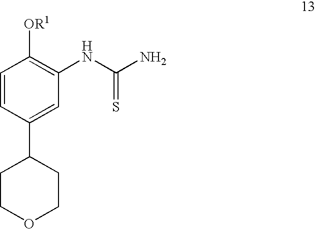
wherein R¹ is alkyl.
